(19) United States
(12) Patent Application Publication
(10) Pub. No.: US 2023/0395243 A1
(43) Pub. Date: Dec. 7, 2023

(54) METHODS AND SYSTEMS FOR UPDATING AND CURATING DATA

(71) Applicant: Evernorth Strategic Development, Inc., St. Louis, MO (US)

(72) Inventors: Jason Teeple, Doylestown, PA (US); Tom R. Loomis, Windham, NH (US); Jonathan E. Magen, Jenkintown, PA (US); Brittany P. DePoi, South Windsor, CT (US)

(21) Appl. No.: 18/204,616

(22) Filed: Jun. 1, 2023

Related U.S. Application Data
(60) Provisional application No. 63/348,037, filed on Jun. 2, 2022.

(51) Int. Cl. G16H 40/20; G16H 10/60

(52) U.S. Cl. CPC G16H 40/20; G16H 10/60; G06Q 40/08

(57) ABSTRACT

Methods and systems for aggregating and curating data are described. In one embodiment, a system comprises a database and a processor configured to (a) receive a trigger generated in response to an individual undergoing a medical event, (b) request data associated with the individual from various sources or channels in response to receiving the trigger, (c) curate data received from the various sources or channels in response to receiving the trigger, and (d) create or append a comprehensive data record based on the curated data received from the various sources or channels

### CROSS-REFERENCE TO RELATED APPLICATIONS

This application claims the benefit of U.S. Provisional Application No. 63/348,037 filed 2 Jun. 2022, the entire disclosure of which is incorporated by reference.

### FIELD

The present disclosure relates generally to the technical field of data processing. In a specific example, the present disclosure may relate to receiving updated data from multiple sources or channels and curating the data for accuracy, recency, and completeness.

### BACKGROUND

Some data is stored in multiple locations across numerous databases resulting in difficulty managing the data, especially if the amount of data is large in scale. Worse yet, when segmented data is constantly refreshed or updated across the multiple locations, managing the data becomes even more difficult. In one example, medical data for a single individual is stored partially by numerous care providers, hospitals, insurance companies, pharmacies, and third-party vendors that manage, host, or participate within medical networks. However, no entity (e.g., care providers, insurance companies, etc.) has a complete and current medical picture of the individual. Typically, medical data is stored in databases as an electronic health record, such as an electronic medical record (EMR), where an EMR is a specific health system's medical record, and an electronic health record includes any health record for a patient including EMRs and records patients can obtain. However, each entity merely stores a respective electronic health record representing the medical data available to each entity, and the electronic health record becomes stale quickly after an appointment or procedure. Indeed, generating a comprehensive patient record providing a complete medical history is very difficult because medical data for the patient is segmented across numerous entities.

### BRIEF DESCRIPTION OF THE DRAWINGS

FIG. 1 is a functional block diagram of an example system according to an example embodiment.

FIG. 2 is a functional block diagram of an example manager device, which may be deployed within the system of FIG. 1.

FIG. 3 is a block diagram of a longitudinal patient record that may be stored within the system of FIG. 1.

FIG. 4 is a block diagram of a flowchart illustrating methods for receiving and curating medical data in response to various trigger messages, according to an example embodiment.

FIG. 5 is a block diagram of a flowchart illustrating methods for generating a completeness score of a longitudinal patient record, according to an example embodiment.

FIG. 6 is a functional block diagram of alternative manager device, which may be deployed within the system of FIG. 1.

FIG. 7 is a pipeline flow diagram of illustrating methods of presenting virtual charts to data consumers, according to an example embodiment.

### DETAILED DESCRIPTION

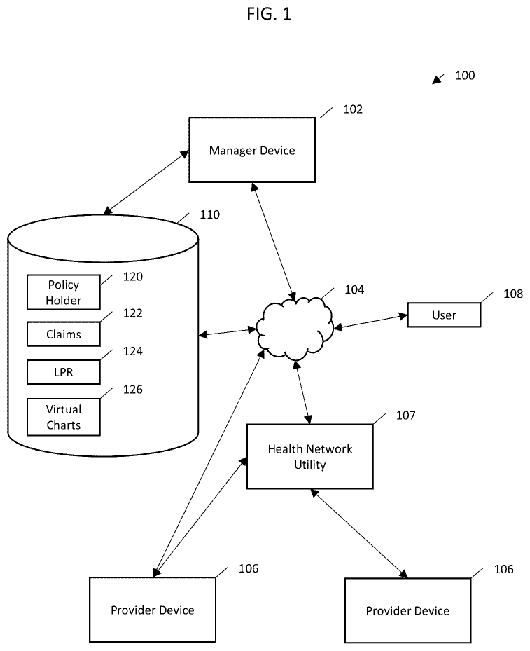

FIG. 1 is a block diagram of an example implementation of a system 100. While the system 100 is generally described as being deployed in a medical context (for example, an insurance company, drug benefit plan manager, etc.), the system 100 and/or components of the system 100 may otherwise be deployed (for example, in a government benefits system, a banking or other financial system, etc.). The system 100 may include a manager device 102, a provider device 106, and a user device 108 in communication with each other directly and/or over a network 104. The system 100 may also include a storage device 110. A single or multiple devices 102, 106, 108, 110 may be used within the system 100.

The manager device 102 is a device operated by an entity that is at least partially responsible for creation and/or management/administration of a health insurance benefit. While the entity operating the manager device 102 is typically an insurance company, other entities may operate the manager device 102 on behalf of themselves or other entities (such as the insurance company). For example, the manager device 102 may be operated by a prescription drug benefit plan, a medical data management company, a retail pharmacy chain, a drug wholesaler, a government branch, a banking institution, a data analytics or other type of software-related company, etc. In some implementations, the insurance company that provides the health insurance benefit may provide one or more than one additional benefits including a medical or health benefit, a dental benefit, a vision benefit, a wellness benefit, a radiology benefit, a pet care benefit, a long-term care benefit, a nursing home benefit, etc. The insurance company may, in addition to its medical insurance operations, operate medical record management and curation on a medical data network, own physician networks or hospitals, or otherwise provide access to medical care. The manager device 102 can include machine executable tasks in memory that can be loaded by a processor for execution, which makes the manager device 102 a dedicated machine to the executable tasks. Examples of such executable tasks are described herein.

Some of the operations of the insurance company that operates the manager device 102 may include the following activities and processes. A policy holder (or a person on behalf of the policy holder) of the medical insurance plan may obtain medical care from a care provider. In some implementations, the policy holder may receive medical care at a medical facility (e.g., surgery at a hospital or an exam at a doctor's office), or the policy holder may receive medical care via a telehealth appointment. Each contact the policy holder has with the medical provider or medical facility generates data that is used by computing machines. The data may include claims data and medical data. The insurance company may fully or partially pay for the medical care depending on various factors. The medical insurance plan is administered by or through the manager device 102, but the manager device 102 may have more functionality or less functionality. In some embodiments, the manager device 102 can perform the methods and functionality described here. In other embodiments, the manager device 102 can include numerous devices capable of performing the methods and functionality described herein as well as all the functionality necessary to administer the health insurance benefit to policy holders, which may include receiving claims, adjudicating them, and providing payment to care providers where applicable.

The user device 108 may be a stand-alone device or may be a multi-use device. Examples of the user device 108 include a set-top box (STB), a receiver card, a mobile telephone, a personal digital assistant (PDA), a display device, a portable gaming unit, and a computing system; however, other devices may also be used. For example, the user device 108 may include a mobile electronic device, such as an IPHONE or IPAD device by Apple, Inc., mobile electronic devices powered by ANDROID by Google, Inc., and a BLACKBERRY device by Research In Motion Limited. The user device 108 also include other computing devices, such as desktop computing devices, notebook computing devices, netbook computing devices, gaming devices, and the like. Other types of electronic devices may also be used. Additionally or alternatively, the user device 108 can execute an application that may use a cellular phone function of the user device 108. The application may include instructions that when executed on the user device 108, in the manager device 102, or one of the one or more than one provider device(s) 106, cause a machine to change its state or perform tasks within the machine and with other machines. The user device 108, the manager device 102 and the provider device 106 when executing instructions to perform tasks can be a dedicated data processing machine for medical related data processing.

A user operating the user device 108 may be receiving a health benefit through a policy. The user may be a policy holder, or may be a spouse, dependent, or caregiver associated with the policy holder. The policy holder may have a copayment for the medical care that reflects an amount of money that the policy holder is responsible to pay the care provider for the medical care of the user. The money paid by the policy holder to the care provider may come from, as examples, personal funds of the policy holder, a health savings account (HSA) of the policy holder or the policy holder's family, a health reimbursement arrangement (HRA) of the policy holder or the policy holder's family, or a flexible spending account (FSA) of the policy holder or the policy holder's family. In some instances, an employer of the policy holder may directly or indirectly fund or reimburse the policy holder for the copayments. In some embodiments, the user operating the user device 108 may be using a discount payment card or payment coupon. In some embodiments, the user operating the user device 108 may be a cash payer not otherwise using a health benefit.

The amount of the copayment required by the policy holder may vary across different medical insurance plans having different plan sponsors or clients and/or for different medical procedures. The policy holder's copayment may be a flat copayment (in one example, $60), coinsurance (in one example, 10%), and/or a deductible (for example, responsibility for the first $5000 of annual medical expense, etc.) for certain medical procedures, certain types and/or classes of medical procedures, and/or all medical procedures. The copayment may be stored in the storage device 110 or determined by the manager device 102.

In some instances, the policy holder may not pay the copayment or may only pay a portion of the copayment for the medical procedure. For example, if a usual and customary cost for a medical office visit is $50, and the policy holder's flat copayment is $60 for medical office visits, the policy holder may only need to pay $50 to undergo the medical office visit appointment. In another example involving a worker's compensation claim, no copayment may be due by the policy holder for the medical procedure.

In conjunction with receiving a copayment (if any) from the policy holder and conducting the medical procedure on the policy holder, the care provider submits a claim to the insurance company for the medical procedure. After receiving the claim, the insurance company (such as by using the manager device 102) may perform certain claim processing and/or adjudication operations including verifying eligibility for the policy holder, determining any appropriate copayment, coinsurance, and deductible for the medical procedure, and performing a medical review for the policy holder. Further, the insurance company may provide a response to the care provider (for example, one of the one or more than one provider device(s) 106) following performance of at least some of the aforementioned operations. In some instances, the adjudication operations may include preauthorizing more expensive medical procedures. In some instances, the claim processing may include processing a claim in accordance with a defined set of applicable claim rules by a claim engine.

As part of the adjudication, the insurance company may ultimately reimburse the care provider for performing the medical procedure when the insurance claim was successfully adjudicated (or deny the insurance claim under certain circumstances resulting in full financial responsibility to the policy holder for the medical procedure). The aforementioned adjudication operations generally occur after the copayment is received and the medical procedure is performed. However, in some instances, these operations may occur simultaneously, substantially simultaneously, or in a different order. In addition, more or fewer adjudication operations may be performed as at least part of the adjudication process.

The amount of reimbursement paid to the care provider by the insurance company and/or money paid by the policy holder may be determined at least partially based on types of insurance networks in which the care provider is included. In some implementations, the amount may also be determined based on other factors. Some or all of the foregoing operations may be performed by executing instructions stored in the manager device 102 and/or an additional device. These can be instructions stored in memory and executed by processors when loaded. The network can include multiple instructions that can be shared by multiple network instruction sets.

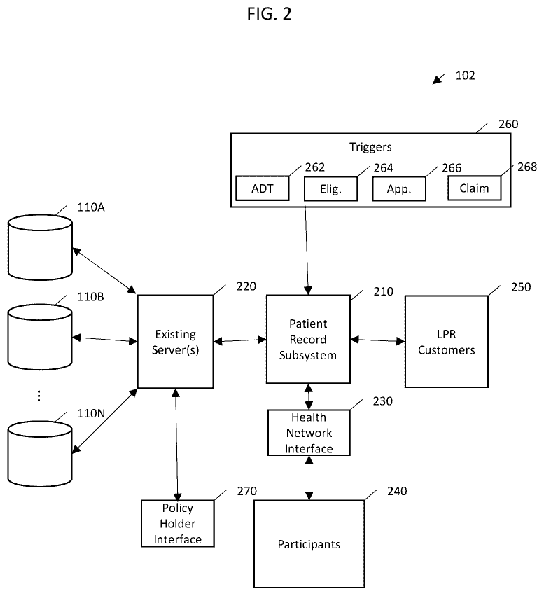

Examples of the network 104 include a Global System for Mobile Communications (GSM) network, a code division multiple access (CDMA) network, 3rd Generation Partnership Project (3GPP), an Internet Protocol (IP) network, a Wireless Application Protocol (WAP) network, or an IEEE 302.11 standards network, as well as various combinations of the above networks. The network 104 may include an optical network. The network 104 may be a local area network or a global communication network, such as the Internet. In some implementations, the network 104 may include a network dedicated to managing medical data: a network such as the electronic network operated by Commonwell, Carequality, or eHealth Exchange. Commonwell can include a primary national network which can connect devices, such as the manager device 102, to regional health exchanges. CareQuality can be an entity that defines protocols and other rules for communicating on medical networks, such as Commonwell. Meanwhile, eHealth Exchange is a government exchange, which also uses CareQuality protocols and rules for communication and data exchange. Network utilities, described with reference to FIG. 2, can help devices, such as the manager device 102, connect to the health networks, such as Commonwell. Network utilities include Health Gorilla, 1Up Health, CareEvolution, Diameter Health, InterSystems, and SureScripts. Data may also come from the user devices 108, either gathered by the user device 108 through, for example, sensors or health tracking apps, or entered by users of the user devices 108. Data may further be provided by payer-to-payer health exchanges.

Moreover, although the system shows a single network 104, multiple networks can be used. The multiple networks may communicate in series and/or parallel with each other to link the devices 102-110.

Each provider device 106 may be a device associated with a care provider or care provider location (e.g., a hospital, a doctor's office, a specialty doctor's office, a pharmacy, medical clinics, testing laboratories, etc.) or other type of medical location at which a policy holder attempts to obtain medical care. The care provider may use the one or more than one of the provider device(s) 106 to submit the claim to the insurance company for adjudication.

Additionally, in some implementations, each provider device 106 may enable an information exchange between the care provider and the insurance company. For example, this may allow the sharing of policy holder information, such as medical history or electronic medical records that may allow the care provider to better service a policy holder (for example, by providing a more informed consultation). Additionally or alternatively, each provider device 106 may respond to various requests from the manager device 102 or other device and provide electronic medical records to the manager device 102 for storage on the storage device 110 or to the other device. In some embodiments, a health network utility 107 existing between the manager device 102 and the one or more than one provider device(s) 106 can gather electronic medical records from all connected provider device(s) 106, thereby generating a network of patient medical data. One such third party vendor or network utility is Health Gorilla, but other medical data vendors or network utilities are contemplated, such as 1Up Health, CareEvolution, Diameter Health, InterSystems, and SureScripts. The health network utility 107 may be excluded in some certain implementations when communication occurs directly between the manager device 102 and the one or more than one provider device(s) 106.

The storage device 110 may include: non-transitory storage (for example, memory, hard disk, CD-ROM, etc.) in communication with the manager device 102 and/or the one or more than one provider device(s) 106 directly and/or over the network 104. The non-transitory storage may store policy holder data 120, claims data 122, longitudinal patient records 124, and virtual charts 126. Further, the system 100 may include additional devices, which may communicate with each other directly or over the network 104.

The policy holder data 120 includes information regarding the policy holders associated with the insurance company. The information stored as policy holder data 120 may include personal information. Examples of the policy holder data 120 include name, address, telephone number, e-mail address, medical history, prescription drug history, demographics, etc. The policy holder data 120 may include a policy holder identifier that identifies the policy holder. The policy holder data 120 may also include preferences such as primary care physicians, preferred specialists, etc. The policy holder data 120 can further identify other beneficiaries of the health insurance benefit associated with the policy holder, such as spouses, dependents, children, etc.

The claims data 122 includes information regarding insurance claims adjudicated by the insurance company as part of the health insurance benefit. In general, the claims data 122 includes an identification of the policy holder that received the medical procedure or other healthcare event giving rise to the claim, the medical procedure or healthcare event that was performed by the care provider (e.g., the medical procedure code number using any acceptable medical coding systems, diagnoses codes using any acceptable medical coding systems, lab test codes using any acceptable medical coding systems, medication codes using any acceptable medical coding systems, etc.), the procedure date, the cost of the medical procedure provided, the copayment/coinsurance amount, and/or policy holder eligibility, etc. Other insurance claims information, as well as other additional information, may also be included in the claims data.

In some implementations, other types of claims beyond insurance claims may be stored in the claims data 122. For example, prescription drug claims, dental claims, wellness claims, or other types of health-care-related claims for policy holders may be stored as a portion of the claims data 122.

In some implementations, the claims data 122 includes claims that identify the policy holders with whom the claims are associated. Additionally or alternatively, the claims data 122 may include claims that have been de-identified (that is, associated with a unique identifier but not with a particular, identifiable policy holder).

The longitudinal patient record (LPR) 124 can include a comprehensive medical profile of the policy holder sourced from numerous care providers, pharmacies, hospitals, third-party medical data vendors, the policy holder data 120, and the claims data 122, etc. The LPR 124 can be a machine readable file that stores data relating to the individual's health. The manager device 102 can create the LPR 124 by compiling or aggregating received medical data (e.g., files) from numerous channels, including data already stored in the storage device 110, as will be described in greater detail below. The LPR 124 can include patient demographics, encounters with care providers, policy holder vital signs gathered by care providers, medications prescribed to the policy holder, immunizations that the policy holder received, procedures performed on the policy holder, test results from tests performed on the policy holder, health goals set by the policy holder or the policy holder's care provider, care plans or medical treatment plans created by care providers for the policy holders, diagnoses or other health conditions of the policy holder, and social determinants of health. The LPR 124 can further include data contributed by a user. For example, data contributed by a user can include information provided from a wearable or other device capable of providing health information. In some embodiments, the user may manually provide health information from an app or a website. The manager device 102 can create the LPR 124 in response to various external trigger messages, as will be described in more detail below. Additionally, the LPR 124 can include a completeness score generated by the manager device 102 or another device connected to the network 104. The completeness score can provide a numerical value indicative of the medical data's comprehensiveness and accuracy, which can provide added confidence to care providers seeking to use the LPR 124 to learn about the patient before a patient arrives for medical care or to guide how medical care is to be administered. For example, an unconscious patient cannot answer medical history questions, so the doctor may rely on an LPR 124 having a high completeness score to make informed medical decisions about the unconscious patient, which may save a patient's life (e.g., the doctor avoids certain medications identified as causing an allergy to the unconscious patient during the course of care). A completeness score for an individual can be based on the particular care path assigned to the patient's LPR 124. In an example embodiment, the completeness score can be weighted to reflect a particular procedure assigned to patient. In some circumstances a first completeness score relates to the overall health records of the patient and a second, specific procedure completeness score is assigned for a particular procedure/medical visit to be undertaken by the patient.

In one particular example, the manager device 102 can create the LPR 124 by aggregating or compiling policy holder data 120, claims data 122, and external data received from the health network utility 107 in response to receiving an accept/discharge/transfer (ADT) message from a care provider on the network 104. In some embodiments, the ADT message comprises Health Level Seven (HL7) data transmitted on the network 104, and the ADT message can represent data associated with numerous medical events including admission of a patient to a care facility, discharging a patient from a care facility, changing an inpatient designation to an outpatient designation or vice versa, changing a patient ID, transferring a patient, or any other ADT message defined by HL7 or other protocol. The present system can be triggered to update the LPR 124 upon an update to the patient record within a provider device or between provider devices. When different devices exchange patient related data, e.g., interface with each other, to send or receive new/updated patient data, that data is sent to the LPR 124.

The virtual charts 126 comprise materialized views of data included in one or more than one LPRs 124. Said differently, each virtual chart 126 can present data included in one or more than one LPRs 124 and present the data in a specialized way according to preferences of a person or device that requested the virtual chart 126. For example, a first doctor may be an allergist, and the allergist may not need all medical data stored in the LPR 124, such as data submitted by an orthopedist, and so the virtual chart 126 presented to the allergist may only contain some of the LPR 124 data that is relevant to treating a patient's allergies. In another example, the manager device 102 may generate a first virtual chart 126 with medical data presented in a table form for a first data consumer (e.g., a first doctor), and the manager device 102 may generate a second virtual chart 126 with the same medical data presented as a graph for a second data consumer (e.g., a second doctor). The manager device 102 may receive preferences from a data consumer, such as one of the provider devices 106 or the user device 108, and generate a virtual chart 126 in response to receiving those parameters or in response to other query information provided at the time the virtual chart 126 was requested. In another embodiment, the preferences may be previously set and stored in the storage device 110. The virtual charts 126 can also change in format depending on the time requested because medical data may have changed between a first virtual chart request and a second virtual chart request.

Example methods and systems for updating and curating data are described. More specifically, example methods and systems for receiving medical data from numerous sources or channels at defined trigger messages or events and curating the data at the defined trigger messages or events to create an LPR 124 are described. Furthermore, example methods and systems for presenting updated and curated data according to user preferences is described. In the following description, for purposes of explanation, numerous specific details are set forth in order to provide a thorough understanding of example embodiments. It will be evident, however, to one of ordinary skill in the art that embodiments of the present disclosure may be practiced without these specific details.

Typically, medical data for an individual is scattered across numerous data storage locations controlled by various entities. For example, a hospital may store an EMR for an individual on a data storage device controlled by the hospital, and the hospital's EMR may store medical data related to an emergency room visit by the individual or surgery performed at the hospital on the individual. Meanwhile, a pharmacy may store prescription drug fulfillment history for the individual on a data storage device controlled by the pharmacy, and a primary care doctor may store vital sign information from yearly physicals administered to the individual on a data storage device controlled by the primary care physician's office. However, in this example, none of the hospital, the pharmacy, or the primary care physician has a complete medical record for the individual. In addition, medical data for the individual may be stale at one or more than one of the above-described entities. For example, the hospital may have vital sign data when a patient had a knee surgery operation in 2015, but the patient may have since developed a high blood pressure condition, rendering the hospital's blood pressure reading stale, outdated, and of little value.

Creating a single electronic health record for an individual reflecting all the medical events associated with the individual can be very difficult. To begin, a single entity would need to receive data from all care providers that provided care to the individual, and the single entity would need to convince every care provider to share this private medical data. Additionally, while medical data networks exist whereby entities, such as insurance companies or other care providers, can request information related to an individual, the information may not have a consistent format, may have incorrect, outdated, or conflicting information, or may simply be too much information for a single computer to manage. Said differently, gathering all EMRs associated with a single individual may not actually provide an accurate or complete health picture of the individual even using the existing networks and medical data sharing providers that currently exist. Thus, there is a need to curate all medical data associated with each individual in order to create a comprehensive and accurate EMR for individuals.

While some methods for curating data might exists, such curation methods are either use case based or constant. Constant medical data curation causes an incredible strain on data computing resources. Such constant medical data curation is costly because it requires frequent requests to either the provider devices 106 or the health network utility 107, and requests to either of those entities may cost money to or impact the resources of the requester. In addition, even if a device associated with the requesting entity were able to make frequent, inexpensive or free requests for medical data, the processing power required to curate massive amounts of data would be unreasonably burdensome, particularly for a significantly large population of individuals, a population size that may be desirable to generate a large enough sample size to run certain analytic processes. For example, an insurance company may have millions of policy holders, and constantly curating medical data for millions of policy holders may not be feasible, efficient, or possible for even modern computing. Use case curation, on the other hand, may not generate an accurate snapshot of an individual's health because the curation is not run at appropriate times or might become stale very quickly. In either situation, better data curation methods are needed in the medical field (or any other field, such as government benefits, financial records, credit reports, etc.)

Finally, even if an entity were able to efficiently curate medical data from numerous sources or channels, the data must still be accurate enough to be useful for data consumers, such as care providers or insurance companies seeking to manage policy holder risk. That is, simply because an entity can combine medical data from multiple sources does not mean that the data is accurate or useful to other data consumers (e.g., doctors, care providers, pharmacies, etc.). Thus, the data must be curated in an effective manner so that the data reflects the most updated, most accurate, and most complete representation of an individual's health. If medical data created from multiple sources were not curated in this manner, the data consumers may not trust the data or may not find it useful for providing care, and the data consumers may try to acquire the medical data through other means (e.g., asking the patient to fill out numerous forms, generating the data themselves, etc.).

At the same time, any medical record must be complete to provide an accurate representation of the medical conditions and care, which requires management of both historical data and current state data. For example, an individual may have developed high blood pressure years ago, but due to diet, exercise, lowered stress, medication, or all the above, the individual no longer suffers from high blood pressure. A complete medical record should store historical data because the individual was, at one time, diagnosed with high blood pressure, and knowing that medical history may be useful for some medical procedures or care. However, the medical record must also recognize the current state of the individual, whereby the individual's vital signs no longer exhibit high blood pressure. Thus, to generate a complete and accurate medical record, a computer would differentiate, categorize, and present data meaningfully, depending on context or preferences, so that a care provider or other data consumer can understand both the patient's medical history and current status. Moreover, data as presented should make sense to the recipient. For example, a patient may not understand medical data in the form of test readings and complicated medical terminology, whereas a doctor would. More granularly, a first doctor may not find all medical data associated with an individual relevant to his, her, or their practice, and so, irrelevant information should not be presented to every care provider. So, data presentation should account for the audience in how it is presented so that the data provided best communicates medical condition to a recipient.

The systems and methods described herein address the above-described issues by curating data in response to meaningful trigger messages. The trigger messages may be related to medical health events, which generate messages on medical networks, such as HL7 messages or any other protocol. For example, the trigger messages can include ADT messages, insurance claim submissions, appointment scheduling, and insurance eligibility submissions. By curating data in response to trigger messages, the processing load on the manager device 102 is reduced so that data curation is only performed at meaningful times. Moreover, the identified trigger messages listed above represent that an individual is about to undergo medical care, is currently undergoing medical care, or recently received medical care, meaning that the trigger represents a significant and meaningful time in the health history of the policy holder. Thus, curating the data at that point indicates that any request made in response to the trigger should result in a complete medical picture for the individual. In response to one of the trigger messages, the systems and methods described herein can receive data from numerous sources, compiling or aggregating the received data, and curate the data, thereby creating an LPR 124.

In addition to receiving and curating data in response to meaningful trigger messages, the systems and methods described herein can generate a completeness score for the curated data indicative of the completeness and accuracy of the created LPR. The completeness score considers expected data, determines any missing data, and also considers ripeness of the data. As part of generating an LPR, the systems and methods can generate a completeness score and associate the completeness score with an LPR 124. Thus, if any care provider or internal system of the manager device 102 requests an LPR 124 or a virtual chart 126, the care provider or internal system of the manager device 102 can take into account the generated completeness score before deciding to use the LPR 124 or virtual chart 126 for medical care or other internal processes.

Moreover, the systems and methods herein generate a curated medical history file (i.e. LPR 124) that combines current medical information and historical medical information after receiving information from numerous sources. In order to generate the LPRs 124, data from the sources is broken down into individual components and then reconfigured based on existing data in the LPR 124, depending on whether the received information provides new data, updated data, or previously unknown data. The curated medical history file furthermore generates a virtual chart for each individual. In addition, the systems and methods can present the virtual chart differently for each data consumer depending on preferences, known information about each data consumer, and other factors so that the data is meaningfully presented and effectively communicated to each data consumer. For example, an individual may receive a virtual chart that is far different in terms of presentation than the virtual chart presented to a doctor at a hospital. In other words, the systems and methods described herein take data from disparate, clinical data sources, harmonize the data from all sources, and aggregate the data into one or more domain-specific models so that data consumers can receive the data needed for their unique purposes.

FIG. 2 is a block diagram of an example architecture implementation of the manager device 102. As shown, the manager device 102 can include a patient record subsystem 210, an existing data server 220, a network adapter 230, participants 240, LPR consumers 250, trigger messages 260, and a policy holder interface 270. While the manager device 102 is generally described as being deployed in a medical context (for example, an insurance company, drug benefit plan manager, etc.), the manager device 102 and/or architectural components of the manager device 102 may otherwise be deployed (for example, in a government benefits system, a banking or other financial system, etc.). The manager device 102 may communicate with the storage device 110 (illustrated in partitions: first partition 110A, second partition 110B, and N^{th} partition 110N).

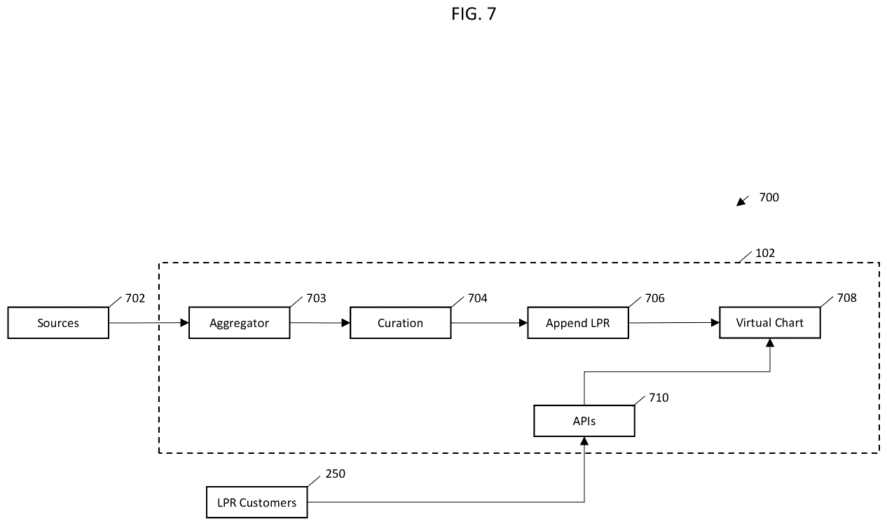

The patient record subsystem 210 is a computer system configured to create LPRs 124 and virtual charts 126 by compiling or aggregating received data. The patient record subsystem 210 can create LPRs 124 by requesting data from various data sources, including the participants 240 and the existing data servers 220. In response to receiving data from the various data sources, the patient record subsystem 210 can combine the data from the various data sources, curate the data, and create the LPR 124 for each participant. The process of generating an LPR 124 and a virtual chart 126 is shown in more detail in FIG. 7. In some embodiments, the existing data servers 220 can provide data stored in the storage device partitions 110A, 110B . . . 110N, data which can include the claims data 122 and the policy holder data 120. Of particular importance may be the claims data 122, as the policy holder data 120 may not be necessary after initially creating an LPR 124. For example, the patient record subsystem 210 may use the policy holder 120 data to assign a policy holder ID to the LPR 124, a number which may not ordinarily change while the policy holder maintains an insurance policy with the insurance company. Additionally, the participants 240 can provide data gathered from various care providers.

The patient record subsystem 210 can curate the data by analyzing the data, ensuring that all data exists in the same format, validating that all received data exists in the correct field, deduplicating the data, and determining timestamp information for each data entry to ensure that the most recent medical data exists in each field. The patient record subsystem 210 may further curate the data by designated data as current or historical and assign data into the LPR 124 accordingly. In some embodiments, the data format for the LPR 124 comprises the Fast Healthcare Interoperability Resources (FHIR) standard, which can include the HL7 protocol. In some embodiments, the patient record subsystem 210 can first gather information for a policy holder from the claims data 122 by communicating with the existing data servers 220 and create a first instance of the LPR 124 using the claims data 122. After creating the first instance of the LPR 124, the patient record subsystem 210 can determine what information (if any) is missing and seek missing information from the participants 240. Additionally or alternatively, the patient record subsystem 210 can validate the claims data 122 using data received from the participants 240, where available, and, in some embodiments, update the claims data 122 using the data received from the participants 240. Alternatively, the patient record subsystem 210 can validate the data received from the participants 240 using the claims data 122.

The patient record subsystem 210 can create or update the LPR 124 in response to trigger messages 260. The trigger messages 260 can include ADT messages 262, insurance eligibility assessment messages 264, appointment scheduling messages 266, and/or insurance claim submissions 268.

In some embodiments, the ADT message comprises Health Level Seven (HL7) data transmitted on the network 104, and the ADT message can represent data associated with numerous medical events including admission of a patient to a care facility, discharging a patient from a care facility, changing an inpatient designation to an outpatient designation or vice versa, changing a patient ID, transferring a patient, or any other ADT message defined by HL7 or other protocol.

An insurance eligibility assessment message 264 can comprise a message sent by a care provider to an insurance company to determine whether a patient remains a policy holder and is covered for certain medical procedures. The insurance eligibility assessment message 264 can include an insurance segment HL7 protocol.

An appointment scheduling message 266 can be a message transmitted in response to a patient scheduling an appointment with a care provider. The appointment scheduling message 266 can include an HL7 Scheduling Information Unsolicited (SIU) message.

The insurance claim submission 268 can include an official insurance claim submitted by a care provider to the insurance company. The insurance claim message 266 can include an HL7 Claim message.

Updating an LPR 124 in response to a trigger 260 can include the patient record subsystem 210 using an append-only change log. An append-only change log may add or supplement information to the LPR 124, but may not delete any information included in the LPR 124. Using the append-only change log may further include reclassifying “current data” as “historical data” in view of newer or more relevant data. For example, in 2020, an individual may have a blood pressure reading taken during a yearly physical at a primary care physician's office, and that blood pressure reading may have been 155/92 mmHg, thereby indicating a high blood pressure diagnosis. However, in 2023, the same individual may have a blood pressure reading taken during a subsequent yearly physical at the primary care physician's office, and that blood pressure reading may have been 128/80 mmHg, thereby indicating that the high blood pressure condition is no longer valid. As a result of the second blood pressure reading, the patient record subsystem 210 may append the LPR 124 by reclassifying the 2020 vitals data as historical data, and also reclassify the high blood pressure diagnosis as historical data as well, and the patient record subsystem 210 may also append the LPR 124 to use the 2023 vitals data as current status. Creation of the append-only change log may involve use of the “upcert” function in SQL when updating the LPR 124 in a database.

In addition, the patient record subsystem 210 may include subsystems to generate the completeness score. Generating the completeness score can include a post-curation service involving appending the LPR to add a data extension according to HL7 protocols. The patient record subsystem 210 can use the claims data 122 stored in the storage device partitions 110A, 110B . . . 110N to determine expected data and possessed data. Based on the ratio of possessed data to expected data, the patient record subsystem 210 can generate a completeness score and assign it to the LPR 124. In addition to this ratio, the patient record subsystem 210 can measure data accuracy by determining how often the claims data 122 matches or substantially matches data received from the participants 240. Moreover, the patient record subsystem 210 can account for data staleness in generating the completeness score. For example, vital sign data over a year old may not have any value in the medical community, and old data may decrease the completeness score. The patient record subsystem 210 can consider data timestamps in determining the data staleness. The completeness score may include a binary number (e.g., 1 for sufficiently complete data, 0 for incomplete data), a percentage score indicating how complete the data is (e.g., 95% complete), a scaled number (e.g., 0 for no data, 10 for a perfectly complete LPR record), or any other scoring method (e.g. letter grades (e.g., “A”, “D”, etc.), a color coding system, emojis, etc.). In some embodiments, a device other than the patient records subsystem generates the completeness score, such as a participant 240, the existing data servers 220, or another device not illustrated in FIG. 2.

In some embodiments, the LPR 124 can have a single completeness score for all instances, or in other embodiments, the LPR 124 can have a completeness score based on use case. For example, some care providers may need a narrow amount of data to provide care, such as an immunologist, who may only need an immunology history to determine whether to administer a vaccination. In this situation, the patient record subsystem 210 may have a robust vaccination history for a patient but lack other important data (such as recent vital sign readings and care plans). In this situation, the patient record subsystem 210 may determine the completeness score in response to receiving a request for the LPR from the immunologist, and the patient record subsystem 210 may generate a very high completeness score because the LPR 124 has the data most important to the immunologist. The patient record subsystem 210 may generate a high completeness score in this situation, even though the completeness score may not be as high generally. A system that determines the completeness score based on use case and in response to a request by a specific care provider improves system performance and reliability because the patient records subsystem can analyze smaller amounts of data and also provide a more accurate completeness score. Also, the processing required to generate the completeness score generally does not occur until necessary. In this embodiment, the completeness score can be generated when generating the virtual charts 126.

In some embodiments, the patient record subsystem 210 can check the completeness score and, if the completeness score is under a certain threshold, the patient record subsystem 210 can initiate an analysis to determine why the data is incomplete. This analysis can include requesting medical data from additional sources (e.g., backup vendors who source medical data, requesting data from the care providers themselves), or the patient record subsystem 210 can determine that data is mislabeled, moved to the wrong field, or some other error. In some situations, the patient record subsystem 210 can notify the providers 240 that the data is incomplete and request updated data.

The patient record subsystem 210 can include various algorithms, including utilization of machine learning, to determine when data is stale or inaccurate. The patient record subsystem 210 can also include intelligence that can investigate the cause why some data appears inaccurate but, in fact, is accurate. For example, the claims data 122 may suggest that a patient has no history of a thyroid condition, while the participant data suggests that the patient has Graves' Disease (e.g., from a diagnosis code included in the participant data). In response, the patient record subsystem 210 may determine that a patient recently saw an endocrinologist and submitted a prescription for Tapazole®. These two events (seeing the endocrinologist and submitting a prescription for a medication associated with the treatment of Graves' disease) can validate that the Graves' Disease diagnosis was not an error. In some embodiments, a device other than the patient records subsystem performs this service, such as a participant 240, the existing data servers 220, or another device not illustrated in FIG. 2.

The patient record subsystem 210 can further generate virtual charts 126 using the LPRs 124 for the LPR customers 250. The virtual charts 126 can present data according to how an LPR customer 250 desires the data to appear. The virtual charts 126 can account for the current data included in the LPR 124, preferences of the LPR customer 250, and data needs of a requesting LPR customer 250. For example, an allergist may not be interested in orthopedic events stored in an LPR 124 for an individual, but the allergist may be very interested in medications that the individual is taking because the individual may be allergic to some medications. The patient record subsystem 210 can generate virtual charts as a function of the generated LPRs 124. The patient record subsystem 210 can update or reformat the virtual chart 126 by accounting for the requesting LPR customer 250 or by receiving preferences parameters through an API used to access the virtual charts 126 by the LPR customer 250. The patient record subsystem 210 may receive the preference parameters prior to generating each virtual chart 126 or at the time an LPR customer 250 requests one of the virtual charts 126. In the example above, the allergist could specify that orthopedic events should be excluded from the virtual chart 126 presented to the allergist, or the patient record subsystem 210 can be programmed to omit orthopedic events from virtual charts presented to any allergist LPR customer 250. Virtual chart 126 presentation can include numerous variables beyond inclusion or omission of medical data included in the LPR 124. For example, the virtual charts may reorder data according to relevance for an LPR customer 250 (e.g., the virtual chart presented to an allergist may list known allergies of the individual first); may present data as a reporting use case or an analytics use case; may present medical data as a document, a table, a graph, or all of the above; may include or omit a photograph of the individual; or any other data presentation change.

The existing data servers 220 can include servers owned and operated by an insurance company in the administration and adjudication of insurance claims. The existing data servers 220 can receive claims submitted by policy holders and care providers and determine an amount of money to be paid to the care providers by the insurance company as part of the health insurance benefit provided to the policy holder. The existing data servers 220 can communicate with the storage device partitions 110A, 110B . . . 110N as part of the process of claim assessment and adjudication. The existing data servers 220 may further communicate with the user device(s) 108 via a policy holder interface 270, which may include a website, a software application, a mobile application, or any other user interface. Policy holders can review claims, update insurance benefits, and perform other interactions with the insurance company via the policy holder interface 270. Additionally, the existing claim servers 220 may provide an LPR 124 or a virtual chart 126 associated with the policy holder to the policy holder via the policy holder interface 270.

The participants 240 can include a medical network comprising numerous entities. The participants 240 can include vendors, such as Health Gorilla, 1Up Health, CareEvolution, Diameter Health, InterSystems, SureScripts, Health IT networks, such as Commonwell, CareQuality, eHealth Exchange, care providers, laboratories, pharmacies, or any other source of medical data. The participants 240 can provide EMRs or other health data in response to requests from the patient record subsystem 210. The patient record subsystem 210 can communicate with the participants 240 via a network adapter 230, which can be a network interface necessary to communicate on various medical data networks. In some embodiments, the network adapter includes encryption methodology and other safeguards to preserve the privacy of medical data as the data is transmitted over a network. The participants 240 can include the provider device 106 and/or the health network utility 107 in FIG. 1.

The LPR consumers 250 include any entity requesting an LPR 124 or a virtual chart 126. The LPR consumers 250 can include care providers, pharmacies, data analytic companies, other insurance companies, network utilities, vendors, health IT networks, or the policy holders themselves. The patient record subsystem 210 can provide the LPR 124 and/or virtual charts 126 to the LPR consumers 250 in response to a request from the LPR consumers 250, which may include determining if the requesting LPR consumer 250 meets the necessary requirements to legally receive private medical data about a policy holder.

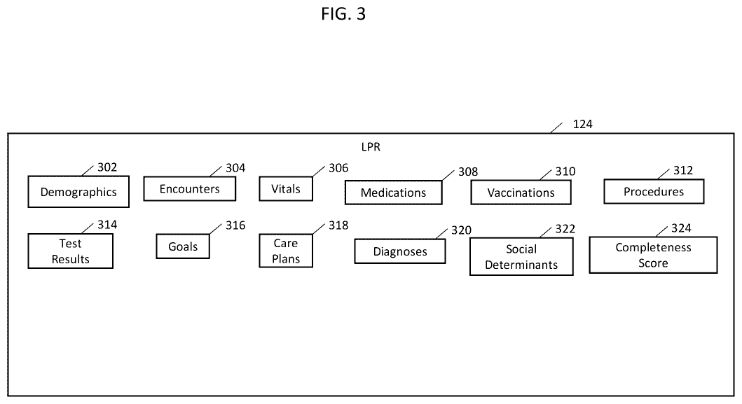

FIG. 3 illustrates an example LPR 124. According to an example embodiment, the LPR 124 can include patient demographics 302, encounters with care providers 304, policy holder vital signs 306, medications prescribed to the policy holder 308, immunizations that the policy holder received 310, procedures performed on the policy holder 312, test results from tests performed on the policy holder 314, health goals set by the policy holder or the policy holder's care provider 316, care plans or medical treatment plans created by care providers for the policy holders 318, diagnoses or other health conditions of the policy holder 320, social determinants of health 322, and a completeness score 324. Additional categories are contemplated, although not illustrated in FIG. 3. While FIG. 3 illustrates a single LPR associated with a single policy holder, it will be understood by those having skill in the art that the patient record subsystem 210 can create and store numerous LPRs where each LPR is associated with a different individual (e.g., policy holders and other members associated with the policy holders). Each of the data categories described above can categorize data as current or historical data, or in some embodiments, only some of the data categories 302-324 classify data as current or historical (e.g., medications, vitals, test results, diagnoses, goals). The data categories can be attributes, data fields, sub-records, metadata, or any other data division within a file or record. Additionally, the data categories discussed above may comply with, follow, or make use of date categories or attributes set by certain data protocols, such as FHIR.

According to an example embodiment, the LPR 124 may comprise data processed such that change to the LPR 124 are immutable. That is, the LPR 124 may comprise only appended data, but data is never removed or deleted after being added to the LPR 124. In this way, the LPR 124 may represent a snapshot of medical data for an individual at numerous points in time, and the status of the individual at many historical points in time are viewable. In this way, a doctor or other medical professional can “go back in time” and see a patient's status at any selected point in historical time after the initial creation of the LPR 124. The system can generate virtual charts 126 that represent any point in time, whether current or historical.

The patient demographics 302 include all known demographics of the patient, including gender, age, ethnicity, marital status, income, employment, education, etc. The patient record subsystem 210 can aggregate or compile the demographics information 302 by receiving the policy holder data 120 and/or data received from care providers or third-party vendors or both.

The encounters with care providers 304 include all known appointments with care providers, telehealth meetings, other visits to a care provider facility, etc. The patient record subsystem 210 can aggregate or compile the encounters with care providers 304 by receiving the claims data 122 and/or data received from care providers or third-party vendors or both.

The policy holder vital signs 306 include gathered vital sign readings including blood pressure, pulse, temperature, respiration rate, oxygen saturation, height, weight, body mass index, etc. The patient record subsystem 210 can aggregate or compile the policy holder vital signs 306 by receiving data from care providers, third-party vendors, or both.

The medications prescribed to the policy holder 308 include known prescriptions prescribed to the policy holder by a care provider based on the data received from the participants 240. The patient record subsystem 210 can aggregate or compile the medications prescribed to the policy holder 308 by receiving the claims data 122 and/or data received from care providers or third-party vendors or both.

The immunizations that the policy holder received 310 include all known vaccinations or other immunization treatments administered to the policy holder by a care provider. The patient record subsystem 210 can aggregate or compile the immunizations that the policy holder received 310 by receiving the claims data 122 and/or data received from care providers or third-party vendors or both.

The procedures performed on the policy holder 312 include known medical procedures administered to the policy holder by a care provider, such as surgeries, blood transfusions, radiation treatments, chemotherapies, physical therapies, etc. The patient record subsystem 210 can aggregate or compile the procedures performed on the policy holder 312 by receiving the claims data 122 and/or data received from care providers or third-party vendors or both.

The test results from tests performed on the policy holder 314 include results from testing performed on the patient, including blood tests, genetic tests, disease tests, etc. The patient record subsystem 210 can aggregate or compile the test results from tests performed on the policy holder 314 by receiving data from care providers or third-party vendors.

The health goals set by the policy holder or the policy holder's care provider 316 include all known health goals, such as weight loss plans, blood pressure aspirations, fitness goals, cancer-free designations, etc. The patient record subsystem 210 can aggregate or compile the health goals set by the policy holder or the policy holder's care provider 316 by receiving the policy holder data 120, the claims data 122, and/or data received from care providers or third-party vendors or all three.

The care plans or medical treatment plans created by care providers for the policy holders 318 include treatment plans prescribed by a care provider, such as cancer treatment plans, blood pressure reduction plans, weight loss plans, reproductive plans, etc. The patient record subsystem 210 can aggregate or compile the treatment plans created by care providers for the policy holders 318 by receiving the claims data 122 and/or data received from care providers or third-party vendors or both.

The diagnoses or other health conditions of the policy holder 320 include known medical diagnoses entered by a care provider, such as genetic disease diagnoses, viral disease diagnoses, high blood pressure diagnoses, acid reflux diagnoses, etc. The patient record subsystem 210 can aggregate or compile the diagnoses or other health conditions of the policy holder 320 by receiving the claims data 122 and/or data received from care providers or third-party vendors or both.

The social determinants of health 322 include known social situations known to be detrimental to health, such as smoking, excessing drinking, drug use, poor working conditions, dangerous jobs or living situations, dangerous recreational activities (e.g., motorcycle driver), etc. The patient record subsystem 210 can aggregate or compile the social determinants of health 322 by receiving the policy holder data 120, claims data 122, and/or data received from care providers or third-party vendors or both.

The completeness score 324 is a score representing how complete and accurate the data in fields 302-322 are. The patient record subsystem 210 can aggregate or compile the completeness score 324 according to the methods and systems described herein. A process for generating the completeness score 324 is described in greater detail below when describing FIG.

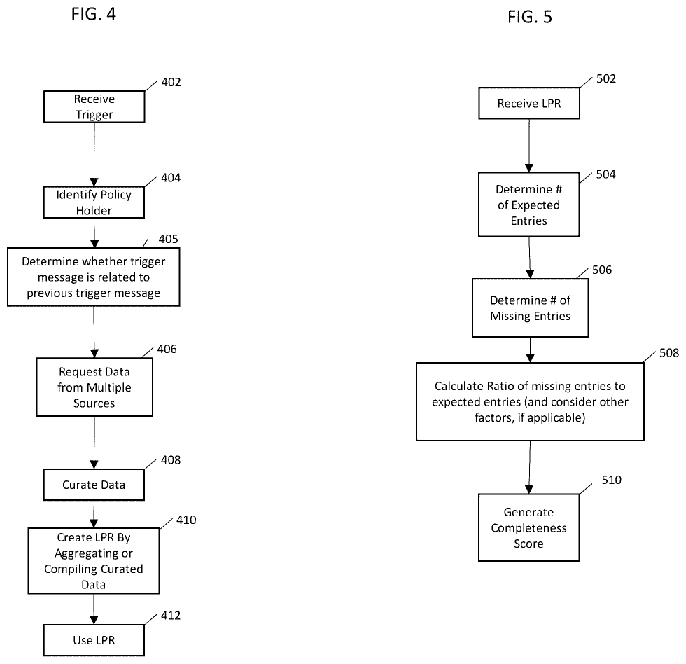

The patient record subsystem 210 can create the LPR 124 in response to a trigger about a policy holder or in response to a policy holder obtaining a health insurance benefit from the insurance company. Moreover, the patient record subsystem 210 can update or re-create the LPR 124 in response to various trigger messages received over a network, including ADTs, insurance eligibility checks, appointment scheduling, etc. A process for creating or updating the LPR 124 is described in greater detail below when describing FIG. 4.

FIG. 4 illustrates a method 400 for receiving and curating medical data in response to various trigger messages according to an example embodiment. The method 400 may be performed by the manager device 102, by the health network utility 107, partially by the manager device 102 and partially by the health network utility 107, or may be otherwise performed. For the sake of simplicity, the manager device 102 will be described as performing the steps of the method 400, but the embodiments described herein are not so limited.

According to an example embodiment, the manager device 102 can receive a trigger message related to a policy holder on a medical network in step 402. According to an exemplary embodiment, the trigger message may comprise ADT messages, insurance claim submissions, appointment scheduling, and/or insurance eligibility submissions. The trigger messages received in step 402 can originate from a care provider having an encounter with a policy holder, such as admitting the policy holder to a hospital, scheduling an appointment with the policy holder, or checking whether a medical procedure will be covered by the policy holder's health insurance benefit.

After receiving the trigger message, the manager device 102 can identify the policy holder based on data included in the trigger message in step 404. In some embodiments, the trigger message can include a patient name or other identifier (e.g., social security number), and the manager device 102 can match the identifier in the trigger message to a policy holder using the policy holder data 120. In some embodiments, the manager device 102 may only look for policy holders in predetermined populations, such as policy holders having a chronic condition. In this example, if the manager device 102 receives a trigger related to a policy holder not within the predetermined population, the method 400 may stop or move back to step 402 to await a trigger related to a policy holder in the predetermined population.

The manager device 102 may also check to determine whether the received trigger is not related to a recently handled trigger in step 405. For example, a care provider may send an ADT trigger and an eligibility check trigger in close succession. In this situation, it may not make sense to continue the method 400 again after the second trigger. Thus, the manager device 102 may determine whether any previous trigger messages related to the policy holder were recently received (e.g., within a predetermined amount of time from the current trigger). If a previous trigger related to the policy holder was recently received (e.g., within the past 15 minutes or 1 hour), the manager device 102 may stop the method 400 because the second trigger is unlikely to result in any updates to the LPR 124. In this way, processing power of the manager device 102 is saved from redundant data gathering and curating steps. In addition to checking the timing of the trigger messages received, the manager device 102 can include smart algorithms or artificial intelligence to ensure that two trigger messages received in close succession are related to the same medical event. For example, the manager device 102 can check the HL7 (or other medical protocol) codes to ensure that the same procedure code or diagnosis code, etc. appears in both trigger messages. Alternatively, the manager device 102 can check the HL7 (or other medical protocol) codes to ensure that both trigger messages relate to the same medical facility or care provider. Other methods of checking that two trigger messages are related are also contemplated.

Subsequently, the manager device 102 can request information about the policy holder from multiple data sources and channels in step 406. In some embodiments, manager device 102 can request medical data about the policy holder from the storage device 100, via the existing data servers 220 (“internal medical data”), and further request medical data about the policy holder from the participants 240 (“external medical data”). In some embodiments, the patient record subsystem device 102 can request the external medical data from a third-party vendor associated with the health network utility 107 or the manager device 102 can request the external medical data from care providers themselves by interfacing with the provider device(s) 106.

After the manager device 102 receives all the data related to the identified policy holder from the various sources and channels, the manager device 102 can curate the data in step 408. Curating the data can include analyzing the data, ensuring that all data exists in the same format, aligning data into the same format (e.g., FHIR), validating that all received data exists in the correct field, deduplicating the data, normalizing the data, and determining timestamp information for each data entry to ensure that the most recent medical data exists in each field. In some embodiments, normalizing the data comprises converting test measurement data into a uniform unit of measurement, converting ID codes in a first medical data format to the data format used by the systems and methods described herein (e.g., FHIR), as well as other data normalizing methods as would be understood by those having skill in the art. In some embodiments, the manager device 102 curates the data, but the example embodiments described herein are not so limiting, and other devices can curate the data. For example, the health network utility 107 can curate the data following instructions from the manager device 102 and after receiving the data from the manager device and/or connected provider device(s) 106. After the data is curated, the manager device 102 can create the LPR 124 by aggregating or compiling the received and curated data in step 410.

Moreover, the manager device 102 can, in some embodiments, use the LPR 124 for subsequent purposes in step 412. The manager device 102 can store the LPR 124 in the storage device 110 for later analysis, the manager device 102 can conduct analysis on the LPR 124 for adjudication or risk management reasons, or the manager device 102 can provide the LPR 124 to eligible recipients. Eligible recipients can include the policy holder herself, the policy holder's primary care physician, a care provider where the policy holder has scheduled an appointment (determined in response to receiving an appointment scheduled trigger), or other departments within the insurance company. In the appointment scheduling example described above, the system and methods described herein exhibit an improvement over the prior art because the care provider will receive updated, curated, and complete data related to the policy holder before the policy holder arrives at the care provider's facility. This situation is particularly useful for a new care provider seeing the policy holder for the first time.

FIG. 5 illustrates a method 500 for generating a completeness score of a longitudinal patient record according to an example embodiment. The method 500 may be performed by the manager device 102, by the health network utility 107, partially by the manager device 102 and partially by the health network utility 107, or may be otherwise performed. For the sake of simplicity, the manager device 102 will be described as performing the steps of the method 500, but the embodiments described herein are not so limited.

According to an example embodiment, the manager device 102 can receive an LPR 124 in step 502. According to an exemplary embodiment, step 502 may occur immediately after step 410 in the method 400 described above, or step 502 can occur in response to an LPR consumer 250 requesting the LPR 124 from the manager device 102, or at any other time.

After receiving the LPR 124, the manager device 102 can determine an expected number of entries in the LPR 124 in step 504 and further determine a number of missing entries in the LPR 124 in step 506. The expected number of entries in the LPR 124 can be determined by analyzing the data received from the claims data 122 and the external medical data (not shown). The manager device 102 can include predictive algorithms that can analyze the received data to determine whether data is missing. For example, if the manager device 102 receives data from a care provider indicating that the policy holder has been diagnosed with cancer (diagnosis data 320), but the LPR 124 contains no care plans or medical treatment plans 318 (such as scheduled radiation or chemotherapy treatments), then the manager device 102 may determine that data is likely missing from the LPR 124. Alternatively, the manager device 102 may determine that the patient was very recently diagnosed with cancer, and so the manager device 102 may determine that the lack of care plans data 318 is not unusual because the doctor has not yet determined the best course of treatment for treating the cancer. In yet another embodiment, the manager device 102 can format a virtual chart 126 to be used for predictive algorithms run by a third party device other than the manager device 102. As illustrated above, the manager device 102 can consider timestamps and data staleness in calculating the completeness score.

After determining the expected number of entries and the number of missing entries, the manager device 102 can determine the ratio of missing entries to expected entries in step 508. The determined ratio can impact the completeness score.

After determining the ratio, the manager device 102 can generate the completeness score in step 510. In some embodiments, the manager device 102 can generate the completeness score based on the determined ratio alone. In another embodiment, the manager device 102 can further determine whether external medical data matches internal data, and when a high amount of external medical data matches internal data, the manager device 102 can increase the completeness score.

In some embodiments, if the completeness score is below a predetermined threshold, the manager device 102 may request external medical data again or request the health network utility 107 update its data from connected care providers. If this process does not result in an improvement to the completeness score, such as by causing the completeness score to exceed the threshold, the manager device 102 may request medical data from care providers directly. The manager device 102 can include intelligence that can target only some care givers and provider devices 106 where missing data is likely to exist, so as to limit the number of network transmissions and data requests. In some embodiments, the manager device 102 may further re-curate the data or analyze the data to determine if data is entered incorrectly into the wrong fields, the result of a translation error (e.g., from one protocol to FHIR), etc.

In some circumstances, the manager device 102 may consider context in generating the completeness score. For example, if a care provider only needs some of the LPR 124 data, and the LPR 124 contains all the necessary data, then the completeness score may be very high as returned to the care provider even if the completeness score is low generally. An example caregiver may be an immunologist who only needs a policy holder's vaccination history. If the LPR 124 contains a robust vaccination history, but lacks other important medical data, the manager device 102 may give the LPR 124 a low completeness score for some caregivers, but a very high completeness score to the immunologist. The manager device 102 may send portions of the LPR 124 to some doctors, depending on the context or use case. In other situations, such as a primary care physician, the manager device 102 may provide the full LPR 124. The completeness score may vary based on a particular inquiry or a particular care provider.

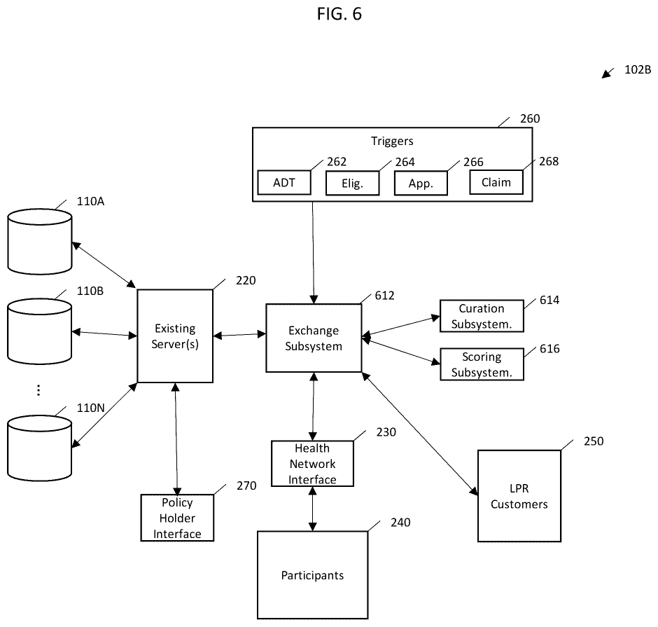

FIG. 6 is a block diagram of an alternative example architecture implementation of the manager device 102B. As shown, and like FIG. 2, the manager device 102B can include the existing data server 220, the network adapter 230, the participants 240, the LPR consumers 250, the trigger messages 260, the policy holder interface 270, and the storage device 110 (illustrated in partitions: first partition 110A, second partition 110B, and N^{th} partition 110N). The functionality of the existing data server 220, the network adapter 230, the participants 240, the LPR consumers 250, the trigger messages 260, the policy holder interface 270, and the storage device 110 is the same in FIG. 6 as it is in FIG. 2.

In addition, the manager device 102B of FIG. 6 includes an exchange subsystem 612, a curation subsystem 614, and a scoring subsystem 616. Together, the exchange subsystem 612, the curation subsystem 614, and the scoring subsystem 616 perform the same tasks and functions as the patient records subsystem 210 of FIG. 2, and FIG. 6 illustrates that the tasks of the patient records subsystem 210 of FIG. 2 may be divided among multiple subsystems.

The exchange subsystem 612 can receive the trigger messages 260 and request data from the participants 240 in response to the trigger messages 260. Upon receiving the data from the participants 240, the exchange subsystem 612 can provide the data for curation to the curation subsystem 614, and the curation subsystem 614 performs the above-described curation functions to curate the received data. The curation subsystem 614 can return the curated data to the exchange subsystem 612, and the exchange subsystem 612 can aggregate and compile the curated data to create the LPR 124 and virtual charts 126. In some embodiments, the curation subsystem 614 can aggregate and compile the curated data to create the LPR 124 and return the LPR 124 to the exchange subsystem 612 for storage, creation of virtual charts 126, or sharing with other entities. The exchange server 612 can further provide the LPR 124 to the scoring subsystem 616, and the scoring subsystem 616 can generate the completeness score according to the methods and processes described above.

FIG. 7 illustrates a flow diagram 700 representing a high-level pipeline for creating an LPR 124 and creating a virtual chart 126 for an LPR customer 250 according to an example embodiment. According to an example embodiment, sources 702 can transmit data to the manager device 102. The sources 702 can include the participants 240 described in FIGS. 2 and 6, the policy holder data 120 described in FIG. 1, the claims data 122 described in FIG. 1, or any other data source. The data transmitted by the sources 702 can comprise FHIR data or data in another format. Data provided in FHIR may have different attributes within it, according to the FHIR protocol, and the FHIR data may be provided to the aggregator 703, which may be the exchange subsystem in FIG. 6, so that the FHIR data may be broken up into logical pieces. For data in another format other than FHIR, the exchange subsystem may also break the data into logical changes within the submitted data according to other format protocol. The exchange manger 703 may request data from the sources 702 in response to trigger messages 260, as described above with reference to FIG. 4.

Upon the aggregator 703 breaking down the data from the sources 702 into logical pieces, the logical pieces are provided to data curation 704. The process of data curation is described above with reference to FIG. 4 (see step 408 in the method 400). The data curation 704 may consider each logical piece and whether the data in each logical piece should be used to append the LPR 124 as in 706. For example, one of the logical pieces provided to curation 704 may be a policy holder's name, which may not have changed. Such data represents duplicate data, and any duplicate data may be disregarded. However, another logical piece may be new vitals data (e.g., new blood pressure reading), and the new vitals data is appended to the LPR 124 as in 706, which may include updating the current status vital data and classifying any other vital data included in the LPR 125 as historical data.

After appending an LPR as in 706, the manager device 102 can generate a virtual chart 126 as in 708. The virtual chart 126 may be a materialized view of the LPR 124, which may be a context-sensitive presentation of some or all of the data included in the LPR depending on a requesting LPR customer 250, which is described in further detail above. The LPR customer 250 may submit a request for the virtual chart 126 through an API 710. The API 710 may receive or have stored preferences of the LPR customer 250, and those parameters are used by the virtual chart creation 708 to determine what data from the LPR 124 to pull and present to the LPR customers 250, which data to omit, and how to present the data to the LPR customer. In some embodiments, the preferences stored in the API are received at the time an LPR customer 250 requests one of the virtual charts 126, or the preferences stored in the API are set prior to generating the virtual chart in step 708. The APIs 710 may also perform the transformations to the presented data that are the virtual charts. Such transformations may occur at query time when an LPR consumer 250 requests the virtual chart or prior to the virtual chart generation 708.

The claimed systems and methods demonstrate an improvement over the prior art. Importantly, the claimed systems and methods can create a longitudinal patient record having complete, comprehensive, and accurate data by gathering data from multiple channels including insurance claims data and EMRs from care providers. Moreover, the claimed systems and methods perform this heavy processing task of gathering medical data from numerous sources and curating the data into an LPR 124 format only at relevant and significant periods of time, such as in response to the trigger messages described above. By limiting when the manager device 102 creates or updates an LPR 124 according to the methods described above, the processing resources of the manager device 102 are efficiently managed while still generating the LPR 124 at appropriate and meaningful instances of time for the policy holder's health and healthcare. Moreover, the completeness score can provide assurance and confidence to care providers who request and subsequently use the LPR 124 for care giving purposes. The completeness score thereby increases the use of LPRs 124 in the medical field, which will improve caregiving by care providers.

In an example, the LPR 124 may trigger a predictive model for testing prior to a medical visit. An example of which is described in U.S. patent application Ser. No. 18/115,835, titled PREDICTIVE MODEL FOR TESTING PRIOR TO VISIT, which is hereby incorporated by reference in its entirety. The predictive model may be executed on the manager device 102 and the testing ordered by the predictive model loaded into the LPR 124, e.g., as part of the encounters 304, medications, 308, procedures 312, test results 314, the like, and combinations thereof. The assignment of testing prior to a visit may affect the completeness score 324.

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. As used herein, the phrase at least one of A, B, and C should be construed to mean a logical (A OR B OR C), using a non-exclusive logical OR, and should not be construed to mean “at least one of A, at least one of B, and at least one of C.”

In the figures, the direction of an arrow, as indicated by the arrowhead, generally demonstrates the flow of information (such as data or instructions) that is of interest to the illustration. For example, when element A and element B exchange a variety of information but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A. The term subset does not necessarily require a proper subset. In other words, a first subset of a first set may be coextensive with (equal to) the first set.

In this application, including the definitions below, the term “module” or the term “controller” may be replaced with the term “circuit.” The term “module” may refer to, be part of, or include processor hardware (shared, dedicated, or group) that executes code and memory hardware (shared, dedicated, or group) that stores code executed by the processor hardware.

The module may include one or more interface circuits. In some examples, the interface circuit(s) may implement wired or wireless interfaces that connect to a local area network (LAN) or a wireless personal area network (WPAN). Examples of a LAN are Institute of Electrical and Electronics Engineers (IEEE) Standard 302.11-2016 (also known as the WIFI wireless networking standard) and IEEE Standard 302.3-2015 (also known as the ETHERNET wired networking standard). Examples of a WPAN are the BLUETOOTH wireless networking standard from the Bluetooth Special Interest Group and IEEE Standard 302.15.4.

The module may communicate with other modules using the interface circuit(s). Although the module may be depicted in the present disclosure as logically communicating directly with other modules, in various implementations the module may actually communicate via a communications system. The communications system includes physical and/or virtual networking equipment such as hubs, switches, routers, and gateways. In some implementations, the communications system connects to or traverses a wide area network (WAN) such as the Internet. For example, the communications system may include multiple LANs connected to each other over the Internet or point-to-point leased lines using technologies including Multiprotocol Label Switching (MPLS) and virtual private networks (VPNs).

In various implementations, the functionality of the module may be distributed among multiple modules that are connected via the communications system. For example, multiple modules may implement the same functionality distributed by a load balancing system. In a further example, the functionality of the module may be split between a server (also known as remote, or cloud) module and a client (or, user) module.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of a non-transitory computer-readable medium are nonvolatile memory devices (such as a flash memory device, an erasable programmable read-only memory device, or a mask read-only memory device), volatile memory devices (such as a static random access memory device or a dynamic random access memory device), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

The computer programs include processor-executable instructions that are stored on at least one non-transitory computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Swift, Haskell, Go, SQL, R, Lisp, Java®, Fortran, Perl, Pascal, Curl, OCaml, Javascript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

What is claimed is:

1. A system comprising:
a database; and
a processor configured to (a) receive a trigger generated in response to an individual undergoing a medical event, (b) request data associated with the individual from various sources or channels in response to receiving the trigger, (c) curate the data received from the various sources or channels in response to receiving the trigger, and (d) create or append a comprehensive data record stored in the database using the curated data.

2. The system of claim 1, wherein the trigger is an admit/discharge/transfer message, an insurance eligibility check message, an appointment scheduling message, or an insurance claim message.

3. The system of claim 2, wherein the admit/discharge/transfer message comprises data associated with admission of the individual to a care facility, discharging the individual from the care facility, changing an inpatient designation for the individual to an outpatient designation for the individual or vice versa, changing a patient ID of the individual, or transferring the individual to another care facility.

4. The system of claim 1, wherein the various sources or channels comprise one or more provider devices associated with one or more medical care providers, a network utility hosting or connected to a network comprising the one or more provider devices, and/or the database.

5. The system of claim 4, wherein the data associated with the individual comprises insurance claims data stored in the database and electronic medical records received from the provider devices or the network utility.

6. The system of claim 1, wherein the comprehensive data record is an append-only change log, and wherein the processor creates or appends the comprehensive data record by adding or supplementing information to the comprehensive data record using the curated data and classifying the information in the comprehensive data record as either current or historical depending on the curated data.

7. The system of claim 1, wherein the processor is further configured to:
determine a number of expected entries in the comprehensive data record and a number of missing entries in the comprehensive data record;
calculate a ratio of the missing entries to the expected entries;
compare the comprehensive data record to insurance claims data stored in the database; and
calculate a completeness score based on the calculated ratio and how many entries in the comprehensive data record match or correspond to data stored in the insurance claims data.

8. The system of claim 7, wherein the processor calculates the completeness score by being further configured to consider data staleness by referencing timestamp data in some or all of the entries of the comprehensive data record.

8. The system of claim 1, wherein curating the data further comprises the processor being configured to analyze the data associated with the individual from the various sources or channels, reformat the data associated with the individual from the various sources or channels into a format of the comprehensive data record, validate that the data associated with the individual from the various sources or channels is placed into a correct field, deduplicate the data associated with the individual from the various sources or channels, and determine timestamp information for each field in the data associated with the individual from the various sources or channels to ensure that the most recent medical data exists in each entry of the comprehensive data record.

9. The system of claim 1, wherein the processor is further configured to:
determine an identity of a data consumer requesting data stored in the comprehensive data record;
determine preferences of the data consumer based on the identity of the data consumer;
gather some or all of the data included in the comprehensive data record depending on the identity of the data consumer or the preferences of the data consumer; and
present the some or all of the data included in the comprehensive data record as a virtual chart according to the preferences.

10. The system of claim 7, wherein the comprehensive data record includes demographics of the individual, encounters with care providers by the individual, the individual's vital signs, medications prescribed to the individual, immunizations that the individual received, procedures performed on the individual, test results from tests performed on the individual, health goals set by the individual or the individual's care provider, care plans or medical treatment plans created by care providers for the individual, diagnoses or other health conditions of the individual, social determinants of health associated with the individual, and the completeness score.

11. The system of claim 1, wherein the processor is further configured to:
compare a timestamp of the trigger and a second time stamp of a second trigger to determine whether the trigger and the second trigger were both received within a time period; and
determine whether a data entry appears in both the trigger and the second trigger;
determine that the trigger and the second trigger are both associated with the medical event when the data entry appears in both the trigger and the second trigger,
wherein the processor does not curate the data when the trigger and the second trigger are both associated with the medical event.

12. The system of claim 11, wherein the data entry is a procedure code, a diagnosis code, a care provider name, or a medical facility name.

13. A method comprising:
receiving a trigger generated in response to an individual undergoing a medical event;
requesting data associated with the individual from various sources or channels in response to receiving the trigger;
curating the data received from the various sources or channels in response to receiving the trigger; and
creating or appending a comprehensive data record stored in the database using the curated data.

14. The method of claim 13, wherein the trigger is an admit/discharge/transfer message, an insurance eligibility check message, an appointment scheduling message, or an insurance claim message.

15. The method of claim 14, wherein the admit/discharge/transfer message comprises data associated with admission of the individual to a care facility, discharging the individual from the care facility, changing an inpatient designation for the individual to an outpatient designation for the individual or vice versa, changing a patient ID of the individual, or transferring the individual to another care facility.

16. The method of claim 13, wherein the various sources or channels comprise one or more provider devices associated with one or more medical care providers, a network utility hosting or connected to a network comprising the one or more provider devices, and/or the database.

17. The method of claim 16, wherein the data associated with the individual comprises insurance claims data stored in the database and electronic medical records received from the provider devices or the network utility.

18. The method of claim 13, wherein the comprehensive data record is an append-only change log, and wherein the processor creates or appends the comprehensive data record by adding or supplementing information to the comprehensive data record using the curated data and classifying the information in the comprehensive data record as either current or historical depending on the curated data.

19. The method of claim 13, further comprising:
determining a number of expected entries in the comprehensive data record and a number of missing entries in the comprehensive data record;
calculating a ratio of the missing entries to the expected entries;
comparing the comprehensive data record to insurance claims data stored in the database; and
calculating a completeness score based on the calculated ratio and how many entries in the comprehensive data record match or correspond to data stored in the insurance claims data.

20. The method of claim 19, wherein calculating the completeness score further comprises considering data staleness by referencing timestamp data in some or all of the entries of the comprehensive data record.

21. The method of claim 13, wherein curating the data further comprises analyzing the data associated with the individual from the various sources or channels, reformatting the data associated with the individual from the various sources or channels into a format of the comprehensive data record, validating that the data associated with the individual from the various sources or channels is placed into a correct field, deduplicating the data associated with the individual from the various sources or channels, and determining timestamp information for each field in the data associated with the individual from the various sources or channels to ensure that the most recent medical data exists in each entry of the comprehensive data record.

22. The method of claim 13, further comprising:
determining an identity of a data consumer requesting data stored in the comprehensive data record;
determining preferences of the data consumer based on the identity of the data consumer;
gathering some or all of the data included in the comprehensive data record depending on the identity of the data consumer or the preferences of the data consumer; and
presenting the some or all of the data included in the comprehensive data record as a virtual chart according to the preferences.

23. The method of claim 19, wherein the comprehensive data record includes demographics of the individual, encounters with care providers by the individual, the individual's vital signs, medications prescribed to the individual, immunizations that the individual received, procedures performed on the individual, test results from tests performed on the individual, health goals set by the individual or the individual's care provider, care plans or medical treatment plans created by care providers for the individual, diagnoses or other health conditions of the individual, social determinants of health associated with the individual, and the completeness score.

24. The method of claim 13, further comprising:
comparing a timestamp of the trigger and a second time stamp of a second trigger to determine whether the trigger and the second trigger were both received within a time period; and
determining whether a data entry appears in both the trigger and the second trigger;
determining that the trigger and the second trigger are both associated with the medical event when the data entry appears in both the trigger and the second trigger,
preventing the curation step when the trigger and the second trigger are both associated with the medical event.

25. The method of claim 24, wherein the data entry is a procedure code, a diagnosis code, a care provider name, or a medical facility name.

26. A system comprising:
a database; and
a processor configured to (a) receive a trigger generated in response to an individual undergoing a medical event, (b) request data associated with the individual from various sources or channels in response to receiving the trigger, (c) receive curated data in response to receiving the trigger, the received curated data being curated by a device separate from the processor using the data associated with the individual from the various sources or channels, and (d) create or append a comprehensive data record stored in the database using the curated data.

27. A method comprising:
receiving a trigger generated in response to an individual undergoing a medical event;
requesting data associated with the individual from various sources or channels in response to receiving the trigger;
receiving curated data in response to receiving the trigger, the received curated data being curated by a device separate from the processor using the data associated with the individual from the various sources or channels; and
creating or appending a comprehensive data record stored in the database using the curated data.
